(19) United States
(12) Patent Application Publication
(10) Pub. No.: US 2017/0128527 A1
(43) Pub. Date: May 11, 2017

(54) PROLACTIN RECEPTOR ANTAGONISTS FOR TREATMENT OF GLIOBLASTOMA

(71) Applicant: ProRec Bio AB, Bromma (SE)

(72) Inventor: Gunnar Norstedt, Bromma (SE)

(73) Assignee: ProRec Bio AB, Bromma (SE)

(21) Appl. No.: 15/317,683

(22) PCT Filed: Jun. 18, 2015
(86) PCT No.: PCT/EP2015/063680

§ 371 Date: Dec. 9, 2016

(30) Foreign Application Priority Data
Jun. 18, 2014 (SE) 1450757-8

(51) Int. Cl. A61K 38/17; A61K 51/10; G01N 33/574; A61K 39/395; A61K 51/08; A61K 45/06; C07K 16/26

(52) U.S. Cl. CPC A61K 38/1709; A61K 47/48246; A61K 47/48415; A61K 45/06; A61K 51/1093; C07K 16/26; A61K 39/3955; A61K 51/088; G01N 33/57492; C07K 2317/76

(57) ABSTRACT

The present inventor has found that glioblastoma cells respond in unique ways to prolactin (Prl) receptor antagonists. The reaction of glioblastoma cells to treatment with Prl receptor antagonists is based on the presence and function of Prl receptors in glioblastomas and the activity can be used for treatment of glioblastomas and other neoplasms of the CNS.

### FIELD OF INVENTION

The present invention relates to the field of treatment of proliferative disorders, in particular treatment of tumours such as glioblastoma, by administration of prolactin receptor antagonists.

### BACKGROUND OF INVENTION

Glioblastomas (ICS; C71.0-C71.9, D43.2) are the most common and the most aggressive primary brain tumors in humans. The incidence is 2-3 cases/100 000 individuals. Treatment involves surgery, chemotherapy and radiation. Without treatment the mean survival time is 4.5 months and with current treatments available this can be extended to 15 month. Because of the severity of the disease, one has tried to find new drugs to treat glioblastomas and this work has e.g. included the identification growth promoting receptors and key signalling systems as well as the search for agents that can block such receptors. As one example one has attempted to block the receptor for platelet derived growth factor (PDGF) and another example concern blocking agents of angiogenesis. Hyper proliferation of glia cells can also be seen in the condition of tuberous sclerosis (ICD Q 85.1) a genetic disease caused by a loss of function of TSC1/TSC2 that regulate the mTOR system.

Prolactin (Prl) is a hormone produced in the pituitary gland. Prl circulates in the blood stream and influences target tissue by binding to a prolactin receptor. A majority of studies on Prl concern actions of tissues outside of the central nervous system (CNS) e.g. breast, prostate and ovary. The existence of a blood brain barrier is considered to prevent entry of Prl into the CNS because of the size of Prl (around 200 amino acids) but it is possible that specific transport systems exist or that Prl can be synthetized within the CNS. There are however relatively few studies in the literature on the Prl system in the brain. There are reports suggesting that Prl receptors are present in glioblastomas (Soares Leaes et al., 2007) and studies also show that addition of Prl stimulates uptake of calcium and proliferation of cancer cells (Ducret et al., 2002, Oliveira-Ferrer et al., 2013). However, a vast amount of receptor types are expressed on glioblastoma cells and therefore the mere presence of Prl receptor per se does not provide any guidance as to its function on glioblastomas.

### SUMMARY OF INVENTION

The present inventors have demonstrated that Prl receptors exist on cultured glioblastoma cells and that addition of exogenous Prl stimulates growth of these cells. Surprisingly, the present inventors also found that Prl receptor antagonists reduce cellular growth. Exposure of glioblastomas for prolactin receptor antagonist provides a novel treatment of glioblastomas.

In a first aspect, the invention concerns a prolactin receptor antagonist for use in the treatment of a neoplasm of the brain and/or spinal cord of a mammal.

In another aspect the invention concerns a method of treatment of glioblastomas of a mammal in need thereof, the method comprising the steps of:

a) obtaining tissue samples of a glioblastoma, and

b) analyzing said sample for presence of Prl receptors,

c) comparing said sample to a control sample from healthy tissue,

d) determining sensitivity of the mammal to treatment with a prolactin receptor antagonist according to any one of the preceding claims,

e) administering a therapeutically effective amount of said prolactin receptor antagonist defined in any one of the preceding claims.

In another aspect the invention concerns a method of inducing cell death in a tumor cell expressing a prolactin receptor, said method comprising administering a prolactin receptor antagonist to a patient diagnosed with a neoplasm of the brain or spinal cord.

In another aspect the invention concerns a method of inhibiting growth and/or invasion and/or proliferation of tumor cells, the method comprising administering a prolactin receptor antagonist to a patient in need thereof.

### DESCRIPTION OF DRAWINGS

FIG. 1: Western blot of glioblastoma cell lines.

The glioblastoma cell line U343MG was tested for prolactin receptor expression using Western blot technique. Expression of Prl receptors were tested in two condition, 10% FCS (NTC) and serum free (S). Three cell lines; U343 MGa, U251 MG were tested for the presence of Prl receptors by Western blot using the antibody (clone 1A2B1, Life Technologies). Antibodies directed against the human Prl receptor detected at least two protein bands of which the larger form (90 kD) is assumed to be the full length receptor. It can be seen that Prl receptors are detectable in glioblastoma cells.

FIG. 2: Immunohistochemistry of Prl receptors in glioblastoma cells. Glioblastoma cells were stained with two different fluorescently tagged anti-Prl receptor antibodies. The two different anti PRLR antibodies that were used were Mouse Monoclonal Antibody MA1-610 (U5), ThermoScientific and Mouse Monoclonal Antibody (clone 1A2B1) (Life Technologies). Cell nuclei were stained with DAPI. The picture shows similar staining pattern of Prl receptors with both antibodies and a strong signal when the antibody (U5) was used.

FIG. 3: Effects of Prl and a Prl receptor antagonist in glioblastoma cells. Glioblastoma cells were grown over night at three different concentration of FCS (0%, 2% and 10%). Cells were then stimulated with Prl (200 ng/ml) or not (control, Ctr) and as indicated cells were exposed to both Prl (200 ng/ml) and the Prl receptor antagonist (PrlR-A) with the following composition; is Prl Δ1-9 S33A, Q73L, G129R, K19OR (SEQ ID NO: 13). After 18 h, crystal violet staining was used to measure proliferation in cultured cells. The upper panel shows results for cultures without FCS, middle panel 2% FCS and lower panel 10% FCS. Bar no1:Control, bar no 2:Prl, bar no 3:Prl receptor antagonist (Cpd51), bar no 4:Prl+Prl receptor antagonist (Cpd51). The antagonist blocked cell proliferation induced by Prl and the effects were most marked at high (10% FCS) serum concentration

FIG. 4: The Prl receptor antagonist blocks Prl induced STATS phosphorylation. Glioblastoma cells were serum starved overnight and were then stimulated with Prl (200 ng/ml) for 15 minutes alone or in combination with different concentrations of the Prl receptor antagonist (Prl Δ1-9 S33A, Q73L, G129R, K19OR i.e. SEQ ID NO: 13). Subsequently, cell extracts were prepared and subjected to Western blotting using antibodies to detect phosphorylated STATS (p-StatS) and GAPDH (FIG. 4a). FIG. 4b shows the effect of different doses of the Prl receptor antagonist (range 40 ng/ml-1000 ng/ml) Total and phosphorylated STAT5 were measured and GAPDH was monitored as an additional control.

FIG. 5: Prl stimulates cell invasion. As demonstrated, treatment with the Prl receptor antagonist (SEQ ID NO: 13) of the invention blocks cell invasion.

### DETAILED DESCRIPTION OF THE INVENTION

Because of the substantial effect of blocking Prl signals on glioblastoma cell growth we claim the use of Prl receptor antagonists for the treatment of glioblastomas. Gliomas are tumors in the brain and spinal cord and glioblastoma tumors can be sub-classified as Astrocytic tumors, Oligodendroglial tumors, Ependymal cell tumors, Mixed gliomas, Neuroepithelial tumors of uncertain origin, Tumors of the choroid plexus, Neuronal and mixed neuronal-glial tumors, Pineal Parenchyma Tumors and Tumors with neuroblastic or glioblastic elements (embryonal tumors). Glioblastomas can also be described based on genetic aberrations and they can also feature stem cell like properties. Tuberosclerosis is not a malignant tumor but this genetic disease has a market feature of glia proliferation.

The prolactin receptor antagonists can either be a monoclonal antibody or ligand based antagonists, optionally modified to change its half-life. In both cases the activation of the Prl receptors is interfered with and a well described activation mechanism is receptor dimerization meaning that two receptors form dimers that activate intracellular signalling systems including the JAK-STAT pathway. The present invention can be practised using different types of Prl receptor antagonists. In one embodiment the antagonists are so called ligand based antagonists using Prl as a back bone, Such antagonist have certain advantages in terms of manufacture, molecular size and may in fact pass the blood brain barrier because their similarity to native Prl. The features of ligand based antagonists include a high affinity for Prl receptor while receptor dimerization is blocked and this defines a class of substances that are useful in the practice of the present invention. This class of substances include the modified Prl designated as Prl Δ1-9 S33A, Q73L, G129R, K190R. This variant has the sequence of native human Prl, Seq ID No1, with the exception that the first 9 amino acids have been deleted and that amino acids in positions 33,73,129 and 190 have been exchanged for A,L,R,R respectively, Seq ID No2. Other Prl modifications of the amino acid sequence in Prl can be made to convert Prl into an antagonist that prevent Prl receptor dimerization and such changes are all within the scope of the present invention if they .lead to substances blocking the Prl receptor.

The Prl receptor antagonists in this invention are so called biological pharmaceuticals composed of specific amino acid sequences. Such agents can be produced using recombinant technologies where genes encoding the desired protein sequences are inserted into a host system that will produce the protein. Commonly used hosts are bacteria and eukaryotic cells. In one embodiment the host for production of the Prl receptor antagonist used in this invention is E. coli but also human eukaryotic cells can be used.

One practice of the present invention is therefore to isolate or synthetize the cDNA encoding human Prl with the modifications required to convert Prl into an antagonist as described above. This gene is inserted into E. coli using a vector that allows the gene to be transcribed and translated into protein. The protein, purified from bacterial extracts, should then be appropriately formulated to become a biopharmaceutical for treatment of glioblastomas.

In the case of monoclonal antibodies, cell clones are isolated that produce antibodies that block Prl receptors, such cells can be expanded and used as a source to purify monoclonal antibodies.

In one embodiment a blocking monoclonal antibody can be used. Such antibodies shall bind the Prl receptor and they may have some sequence similarity to the binding of Prl to its receptor. It is therefore possible to use the information stated above to create antibody-like molecules blocking the Prl receptor. Alternatively it is possible to screen for new antibodies. The reagents needed for screening is a recombinant E. coli produced Prl receptor consisting of cDNA encoding the extra cellular domain of the receptor. It is also required to have access to recombinant or purified Prl in order to set up an assay measuring binding of Prl to its receptor. Such assays can be designed in many different ways. There are also different methods to screen for monoclonal antibodies. One principle has been to create monoclonal antibodies in animals using the immune response to identify antibodies interfering with Prl binding and they “humanize” an isolated antibody using techniques of molecular biology. An alternative is to directly screen a library consisting of human antibody genes which can be expressed and tested for blocking the binding between Prl and the Prl receptor.

In one embodiment, Prl receptor gene expression is silenced using anti-sense DNA or siRNA. The design of such molecules originates from the Prl receptor gene sequence: Prolactin receptor (PrlR) NCBI gene ID 5618. Procedures to silence gene expression of the Prl receptor include the use of anti-sense DNA, siRNA or microRNA. Delivery of such gene silencing reagents can include viral or chemical transfection procedures.

In the field of protein therapy it is well known that the excipient is of large value to preserve stability, shelf life and bioactivity. The present invention therefore includes the use of different excipients ranging from amino acids e.g. glycine to carbohydrates e.g. mannitol that can be used to formulate the antagonist in an acceptable formulation to be injected into a living organism.

The present invention concerns treatment of subjects with glioblastomas with a Prl receptor antagonist and such treatments include different modes of administration. The antagonist can be administered via any suitable route such as by subcutaneous injections but it can also be by intravenous or intra-thecal delivery or directly onto the tumor site. The amount to be injected will vary but should be sufficient to block Prl receptors.

A factor of significance is further the pharmacokinetic profile of the biopharmaceutical to be injected. There are different means to change the half-life of proteins and a commonly used procedure is to PEGylate the protein of interest. An alternative it is create conjugates to albumin or to fuse the protein of interest to the FC portion of antibodies. In the case of Prl receptor antagonists for treatment of glioblastomas the need to change half-life will depend on the route of administration and the type of tumor to be treated.

In one embodiment the antagonist is subcutaneously injected into a patient with a glioblastoma but other modes of delivery can be considered including intravenous, intrathecal and directly on the tumor site. The dose of treatment can vary between e.g. 1-300 mg/day such as 10-30 mg/day. In one embodiment the drug composition is formulated as a lyophilized powder reconstituted before injection. The duration of treatment will also vary and is likely to be individually determined by the treating doctor. One key determinant is how the tumor size is affected by the treatment which can be determined by using different imaging techniques in standard clinical use.

It is also to be stated that treatment using the Prl receptor antagonist may be combined with other drugs for the treatment of glioblastomas and that combination treatment can improve the treatment outcome. Prl receptor antagonists affect a specific signalling pathway that does not overlap with other pathways. Therefore drugs affecting other pathways of relevance for glioblastoma treatments can be combined with treatments using a Prl receptor antagonist. Examples of such treatments include compounds affecting signals related to PDGF, EGF, angiogenic factors, kinase inhibitors such as staurosporine and mitogenic blockers such as Docitaxel.

The above mentioned antagonist, Prl Δ1-9 S33A, Q73L, G129R, K19OR (SEQ ID NO. 13): works by blocking Prl receptor dimerization but the ability to do this is not unique to this specific molecule. In fact other molecules e.g. monoclonal antibodies block Prl receptors in a similar manner and principally one can also use low molecular weight compounds to block the Prl receptor although such are not available yet. It is also possible to reduce the level of Prl receptor gene expression and for this the terms siRNA or antisense DNA are well known for persons skilled in the art. In terms of reducing growth of glioblastomas we predict that any substance with the ability to block Prl receptors will have similar effects. Therefore any substance blocking the Prl receptor can be used to affect glioblastoma growth. In one embodiment the use of a ligand based antagonist is Prl Δ1-9 S33A, Q73L, G129R, K19OR (SEQ ID NO. 13), optionally modified to increase its half-life when injected into an organism. The means to extend half-life of proteins can be PEGylation or linking the protein to albumin but other methods are known to persons skilled in the art. In the particular case of treating brain tumors it is essential to reach a sufficiently high concentration at the site of the tumor. Mechanisms to transport Prl into the CNS may be the function of Prl receptor levels in the choroid plexus and therefore ligand based Prl receptor antagonists may enter CNS via such receptors

In certain embodiments, the present invention is as described in the claims as originally filed.

### EXAMPLES

### Example 1

### Preparation of a Prl Receptor Antagonist

Human Prl cDNA was obtained from commercial sources (Sino Biological Inc., Beijing China). The amino acid sequence in Prl cDNA was then be altered by site directed mutagenesis by using kits available from several vendors. The entire cDNA sequence can also be synthetized using services from e.g. Cambridge Bio Science. Ltd (Cambridge UK). The cDNA sequence encoding the polypeptide Prl Δ1-9 S33A, Q73L, G129R, K19OR (SEQ ID NO: 9) was put into a bacterial expression vector pNIOC28-Bsa4. Following introduction into E. coli (DH5 alpha). Other vectors can also be used. The His tagged protein was purified using Ni columns. Alternative modes of purification with or without purification tags can be utilized.

### Example 2

### Expression of Prl Receptors in Cultured Glioma Cells

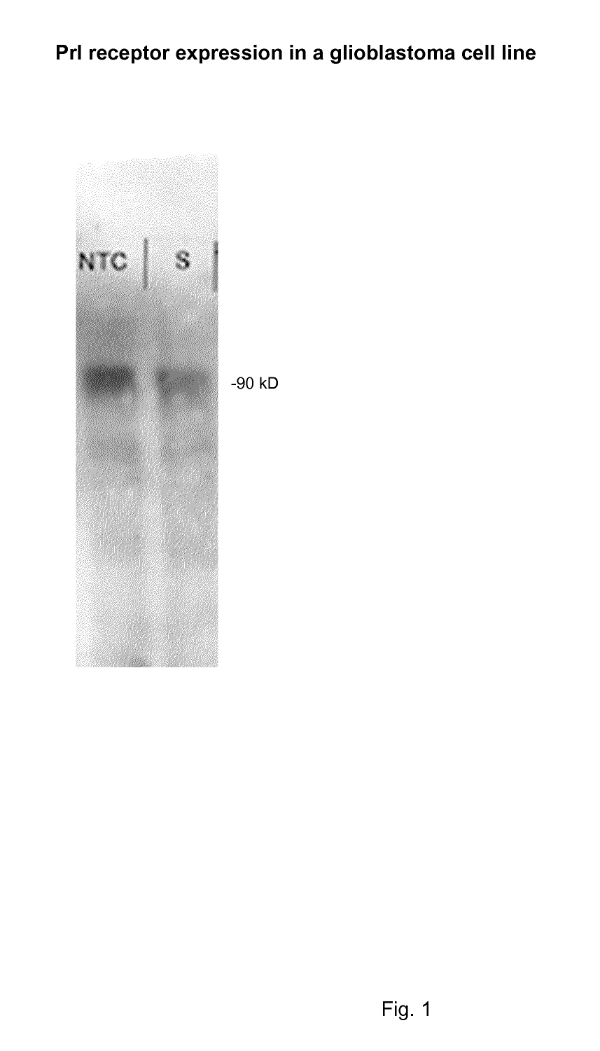

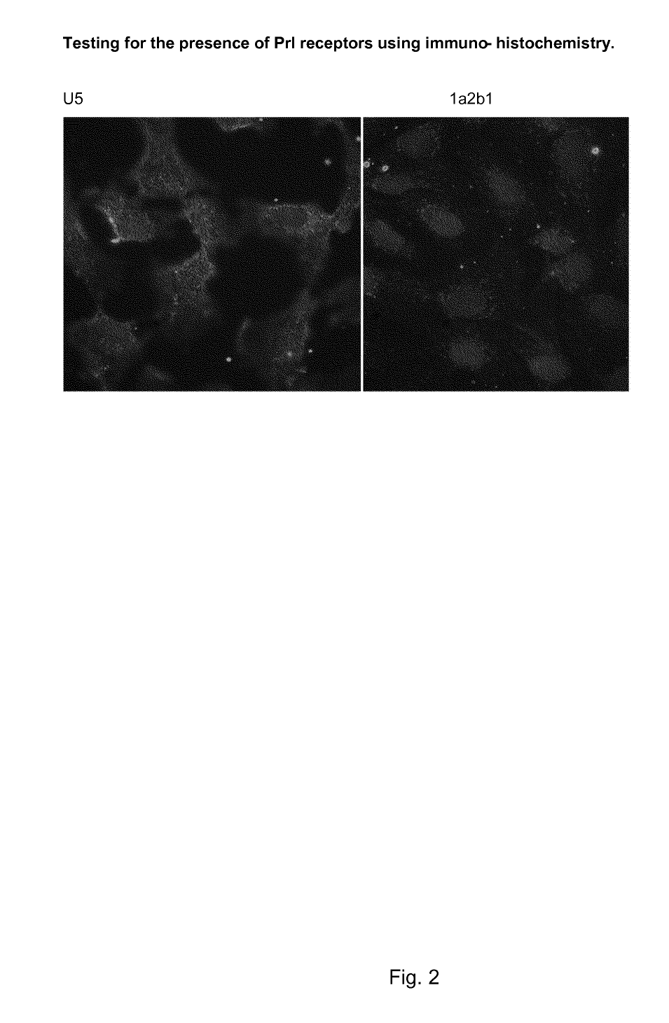

Human glioblastoma cells can be obtained from different sources including ATCC. The tested cell line were shown to express Prl receptors using both Western blots and immunohistochemistry and both methods are well established procedures to detect Prl receptors. A prerequisite for the tumors to respond to Prl receptor antagonist treatment is the presence of Prl receptors on tumor cells or on adjacent cells. As demonstrated in FIGS. 1 and 2, Western blot or immunohistochemistry can be used to detect the presence of Prl receptors in such cells.

### Example 3

### Glioblastoma Cells Respond to Added Prl by Proliferation

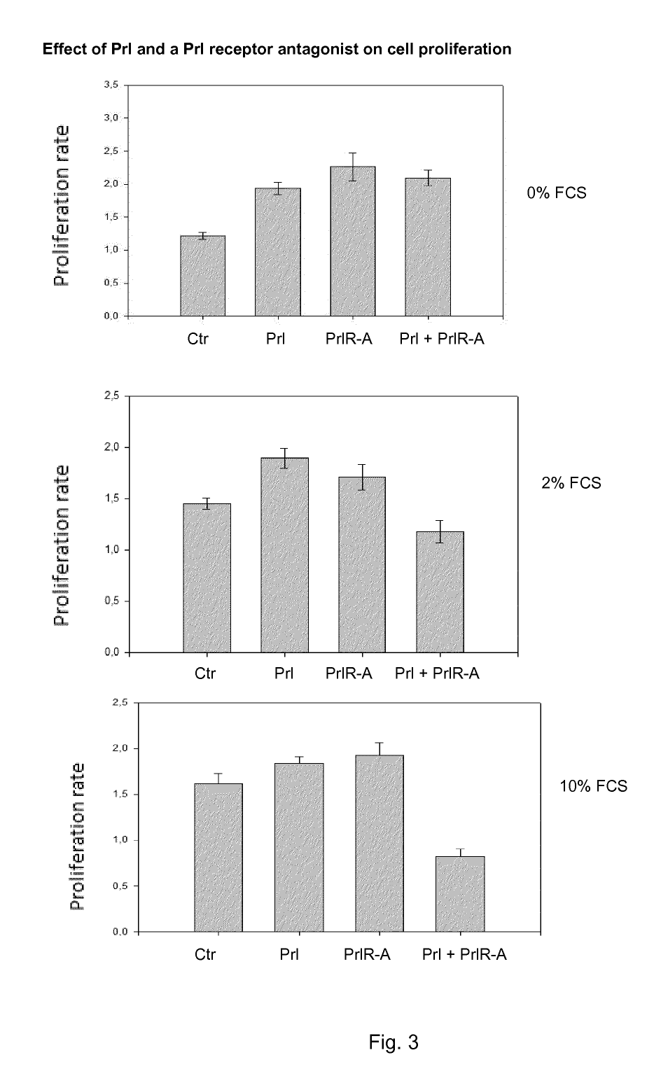

The read-out to measure proliferation in this case was based on the ability of crystal violet to stain cells but other techniques to measure cell proliferation can be used. The experiment in FIG. 3 shows that the Prl receptor, present on glioma cells, is biologically active. It also shows that the effect of Prl is most marked in serum starved cells whereas the effect in 10% serum is not so pronounced.

### Example 4

### Addition of a Prl Receptor Antagonist Blocks Cell Growth

Surprisingly, the effect of blocking Prl was most dramatic in the presence of fetal calf serum (FCS) as demonstrated in FIG. 3. The present experiments indicate that FCS contains Prl-like molecules/components involved in Prl actions or can induce synthesis of Prl in human cells. The finding that the Prl receptor antagonist (SEQ ID NO: 13) can block cells under optimal growth condition i.e. 10% serum, regardless of the mechanism involved is a key finding in terms of the use of the Prl receptor antagonist in the treatment of glioblastomas.

### Example 5

### The Prl Receptor Antagonist Blocks Cell Signalling

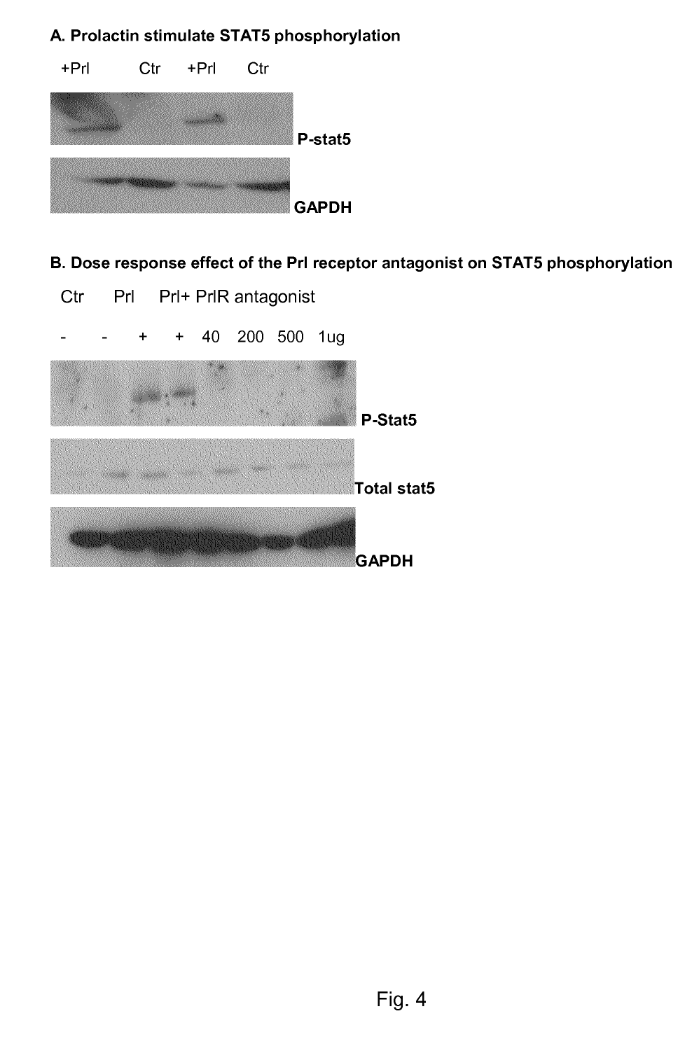

In terms of signals that are Prl dependent in glioblastoma cells, we found that addition of exogenous Prl activates (phosphorylates) the JAK-STAT5 pathway and that this effect is blocked by the Prl receptor antagonist (FIG. 4). Signal transducer and activator of transcription 5 (STAT5) is a transcription factor that is important for cellular growth in certain cells. In this example cells were cultured in Dulbecco's Modified Eagle's Medium (DMEM) supplemented with 10% Fetal Bovine Serum (FBS) (Gibco),100 U/ml penicillin and 100 μg/ml streptomycin at 37° C., 5% CO2. The cells were cultured without serum over night and were then stimulated with Prl (200 ng/ml) for 15 minutes. This stimulation was performed with or without different concentrations (40 ng/ml −1 ug/ml) of the Prl receptor antagonist Prl Δ1-9 S33A, Q73L, G129R, K19OR (SEQ ID NO: 13). Cells were lysed in 50 mM Tris HCl, pH 7.5/150 mM NaCl/5 mM EDTA/0.5% Igepal-40/1 mM Na3VO4/20 mM NaF/1 mM DTT/1 mM PMSF/1× Cocktail inhibitor (Complete mini, Roche). Cell debris was removed by centrifugation at 14,000×g for 15 minutes at 4° C. PRL hormone treatment concentration was 200 ng/ml unless otherwise specified. The protein content of the supernatant was determined using the Bradford dye-binding method.

Whole cell lysates were separated in SDS/PAGE gels and transferred to polyvinylidenediflouride (PVDF) membranes (Millipore). After blotting membranes were blocked in 5% non-fat skim milk or BSA (Sigma) in Tris-Buffered Saline (TBS) containing 0.1% Tween 20. Membranes were incubated with one or more of the following antibodies; PrlR antibody clone 1A2B1 (Invitrogen Thermo Ficher Scientific Waltham Mass.)). Antibodies to detect phosphorylated and un-phosphorylated STAT5 and STAT3 were obtained from Cell Signalling Technology (Danvers Mass.). For loading control, antibodies detecting GAPDH were used. Horse-radish peroxidase (HRP) conjugate secondary antibodies (Cell Signalling or Santa Cruz) were used for detection. Membranes were visualized with the ECL Western blotting detection system (Pierce) according to the manufacturer's instruction or Amersham ECL Prime Western Blotting Detection Reagent from GE healthcare. In essence we think that we have identified a model system where Prl receptor antagonists can be studied and that blocking of Prl receptors have a future medical utility for the treatment of glioblastomas.

### Example 6

### Prolactin Receptor Antagonist in a Clinical Setting

A 45-year-old man suffers from fatigue, morning headache and slurred speech. In the medical center, MRI identifies a froto-parietal lesion with edema in the right hemisphere. The patient is transferred to the neurosurgery department where the lesion is steriotactically removed resulting in subtotal resection of the lesion. Subsequent pathological analysis reveal a glioblastoma multiformi (GMB).

Immunohistochemistry is also performed to analyse several markers for GMB. This analysis also include the analysis of the prolactin receptor which is found to be elevated.

In the post-operative phase the patient respond poorly to conventional medication for which reason a treatment with a prolactin receptor antagonist is initiated. The Prl receptor antagonist is injected subcutaneously at daily intervals using a single loading dose of 40 mg followed by daily injections of 10 mg. The patient is monitored regularly and clear signs of a reduced tumor expansion is subsequently demonstrated.

### Example 7

### PrlR is Expressed in Different Brain Tumors

A tissue micro array (TMA) was purchased from Biomax Inc (Rockville, Md. 20850, USA). This TMA contains samples (histological sections) from 78 different cases of brain tumors (glioblastomas, astrocytomas, ependymomas, oligo-astrocytomas medulloblastoma and oligodentrogliomas). Immunohistochemistry was conducted to detect the human Prl receptor and demonstrated that the receptor was detectable in different types of brain tumors. The experiment thus shows that the Prl receptor is expressed in different forms of human brain tumors and is a suitable target for Prl antagonists of the present invention.

### Example 8

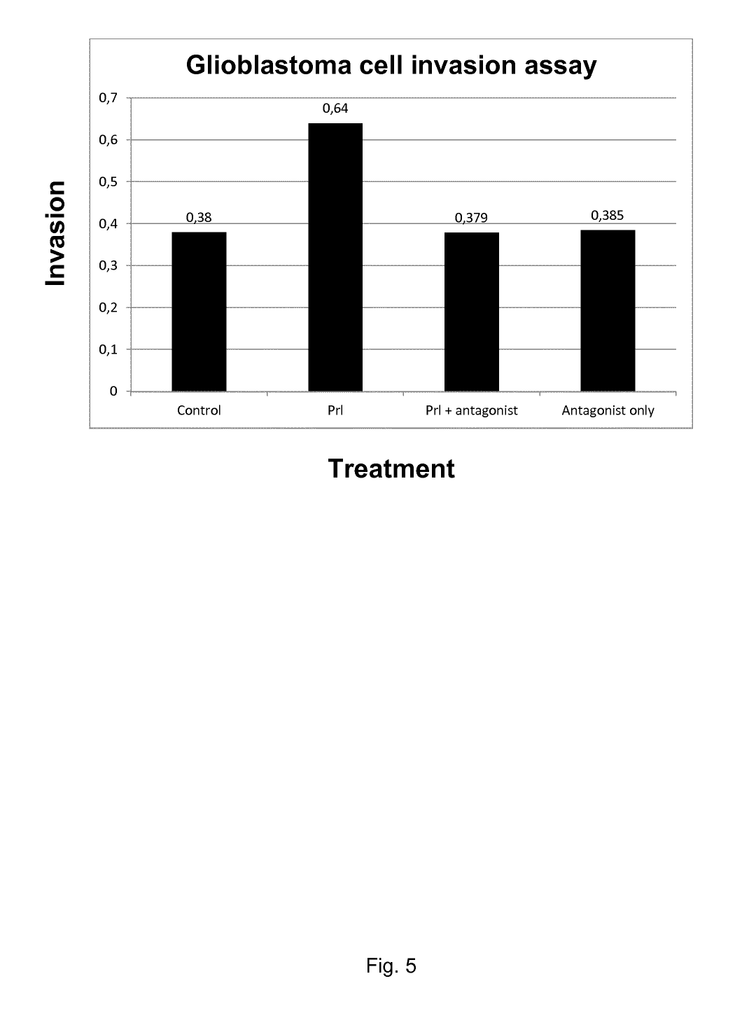

The glioblastoma cell line U251 MG was starved overnight. Prolactin (200 ng/ml) was added over night with or without simultaneous addition of the Prl receptor antagonist (SEQ ID NO: 13; 200 ng/ml) and control cells were exposed to vehicle. The invasive properties of tumor cells were analyzed using CytoSelect™ Cell Invasion Assay kit (Cell Biolabs, Inc., San Diego, Calif.), according to the manufacturer's instructions. The optical density of stained invading cells were measured at 560 nm. The invasive properties of human U251 MG cells cells were increased by the addition of hPrl and the increased invasion was blocked by a simultaneous addition of the Prl receptor antagonist. Under the conditions used, the high affinity PrlR antagonist added on its own did not affect cell invasion (see Table 1/FIG. 5).

TABLE 1

| Treatment | Invasion (AU) |
|---|---|
| Control | 0.380 |
| Prl | 0.640 |
| Prl + Prl receptor antagonist | 0.379 |
| Prl receptor antagonist | 0.385 |

### REFERENCES

DUCRET, T., BOUDINA, S., SORIN, B., VACHER, A. M., GOURDOU, I., LIGUORO, D., GUERIN, J., BRESSON-BEPOLDIN, L. & VACHER, P. 2002. Effects of prolactin on intracellular calcium concentration and cell proliferation in human glioma cells. Glia, 38, 200-14.
OLIVEIRA-FERRER, L., WELLBROCK, J., BARTSCH, U., PENAS, E. M., HAUSCHILD, J., KLOKOW, M., BOKEMEYER, C., FIEDLER, W. & SCHUCH, G. 2013. Combination therapy targeting integrins reduces glioblastoma tumor growth through antiangiogenic and direct antitumor activity and leads to activation of the pro-proliferative prolactin pathway. Mol Cancer, 12, 144.
SOARES LEAES, C. G., FILHO, A. P., PEREIRA LIMA, J. F., DALLAGO, C. M., BATISTA, R. L., BARBOSA-COUTINHO, L. M., FERREIRA, N. P. & DA COSTA OLIVEIRA, M. 2007. Hyperprolactinemia and immunohistochemical expression of intracellular prolactin and prolactin receptor in primary central nervous system tumors and their relationship with cellular replication. 2.

### SEQUENCE OVERVIEW

SEQ ID NO. 1: Human Prolactin Receptor (PrlR)

SEQ ID NO. 2: Human Prl including signal peptide (wild-type)

SEQ ID NO. 3: Human mature Prl (wild-type)

SEQ ID NO. 4: Human mature Prl (mutated 533A, Q73L, G129R, K190R)

SEQ ID NO. 5: Human N-terminally truncated (Δ1) Prl (mutated 533A , Q73L, G129R, K190R)

SEQ ID NO. 6: Human N-terminally truncated (Δ1-2) Prl (mutated 533A, Q73L, G129R, K190R)

SEQ ID NO. 7: Human N-terminally truncated (Δ1-3) Prl (mutated 533A, Q73L, G129R, K190R)

SEQ ID NO. 8: Human N-terminally truncated (Δ1-4) Prl (mutated 533A, Q73L, G129R, K190R)

SEQ ID NO. 9: Human N-terminally truncated (Δ1-5) Prl (mutated 533A, Q73L, G129R, K190R)

SEQ ID NO. 10: Human N-terminally truncated (Δ1-6) Prl (mutated 533A, Q73L, G129R, K190R)

SEQ ID NO. 11: Human N-terminally truncated (Δ1-7) Prl (mutated 533A, Q73L, G129R, K190R)

SEQ ID NO. 12: Human N-terminally truncated (Δ1-8) Prl (mutated 533A, Q73L, G129R, K190R)

SEQ ID NO. 13: Human N-terminally truncated (Δ1-9) Prl (mutated 533A, Q73L, G129R, K190R)

SEQ ID NO. 14: Human N-terminally truncated (Δ1-10) Prl (mutated 533A, Q73L, G129R, K190R)

SEQ ID NO. 15: Human N-terminally truncated (Δ1-11) Prl (mutated 533A, Q73L, G129R, K190R)

SEQ ID NO. 16: Human N-terminally truncated (Δ1-12) Prl (mutated 533A, Q73L, G129R, K190R)

SEQ ID NO. 17: Human mature Prl (mutated 561A, D68N, Q73L, G129R, K190R)

SEQ ID NO. 18: Human N-terminally truncated Prl (Δ1) (mutated 561A, D68N, Q73L, G129R, K190R)

SEQ ID NO. 19: Human N-terminally truncated Prl (Δ1-2) (mutated 561A, D68N, Q73L, G129R, K190R)

SEQ ID NO. 20: Human N-terminally truncated Prl (Δ1-3) (mutated 561A, D68N, Q73L, G129R, K190R)

SEQ ID NO. 21: Human N-terminally truncated Prl (Δ1-4) (mutated 561A, D68N, Q73L, G129R, K190R)

SEQ ID NO. 22: Human N-terminally truncated Prl (Δ1-5) (mutated 561A, D68N, Q73L, G129R, K190R)

SEQ ID NO. 23: Human N-terminally truncated Prl (Δ1-6) (mutated 561A, D68N, Q73L, G129R, K190R)

SEQ ID NO. 24: Human N-terminally truncated Prl (Δ1-7) (mutated 561A, D68N, Q73L, G129R, K190R)

SEQ ID NO. 25: Human N-terminally truncated Prl (Δ1-8) (mutated 561A, D68N, Q73L, G129R, K190R)

SEQ ID NO. 26: Human N-terminally truncated Prl (Δ1-9) (mutated 561A, D68N, Q73L, G129R, K190R)

SEQ ID NO. 27: Human N-terminally truncated Prl (Δ1-10) (mutated 561A, D68N, Q73L, G129R, K190R)

SEQ ID NO. 28: Human N-terminally truncated Prl (Δ1-11) (mutated 561A, D68N, Q73L, G129R, K190R)

SEQ ID NO. 29: Human N-terminally truncated Prl (Δ1-12) (mutated 561A, D68N, Q73L, G129R, K190R)

SEQ ID NO. 30: Human N-terminally truncated S-Prl (Δ1-12) (mutated 561A, D68N, Q73L, G129R, K190R)

SEQ ID NO 31: CPGPPGS (N-terminal tag)

SEQ ID NO 32: 3) DDEWLCGWRPLCIDEILRPGPPGS (N terminal albumin binding peptide)

SEQ ID NO. 33: Prl—Human N-terminally truncated (Δ1-9) Prl (mutated 533A, Q73L, G129R, K190R) with N-terminal Serine i.e. Ser-SEQ ID NO.13).

SEQ ID NO. 34: Prl—Human N-terminally truncated Prl (Δ1-9) (mutated 561A, D68N, Q73L, G129R, K190R) with N-terminal Serine i.e. Ser-SEQ ID NO.26)

1. A prolactin receptor antagonist for use in the treatment of a neoplasm of the brain and/or spinal cord of a mammal.

2. The prolactin receptor antagonist for use according to claim 1, wherein the neoplasm is a malignant neoplasm.

3. The prolactin receptor antagonist for use according to any one of the preceding claims, wherein the neoplasm is a glioblastoma.

4. The prolactin receptor antagonist for use according to any of the preceding claims wherein the neoplasm of the brain and/or spinal cord is selected from the group consisting of Astrocytic tumors, Oligodendroglial tumors, Ependymal cell tumors, Mixed gliomas, Neuroepithelial tumors of uncertain origin, Tumors of the choroid plexus, Neuronal and mixed neuronal-glial tumors, Pineal Parenchyma Tumors and Tumors with neuroblastic or glioblastic elements (embryonal tumors).

5. The prolactin receptor antagonist for use according to any one of the preceding claims, wherein the antagonist is selected from the group consisting of:
a) a polypeptide
i) comprising or consisting of an amino acid sequence selected from the group consisting of SEQ ID NO: 13, SEQ ID NO: 33, SEQ ID NO: 26, SEQ ID NO: 34, SEQ ID NO: 4, SEQ ID NO: 5, SEQ ID NO: 6, SEQ ID NO: 7, SEQ ID NO: 8, SEQ ID NO: 8, SEQ ID NO: 9, SEQ ID NO: 10, SEQ ID NO: 11, SEQ ID NO: 12, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 27, SEQ ID NO: 28, SEQ ID NO: 29 and SEQ ID NO: 30, or
ii) a biologically active variant of i), wherein the variant comprises a sequence which is at least 70% identical to, such as at least 75% identical to, such at least 80% identical to, such at least 85% identical to, such at least 86% identical to, such at least 87% identical to, such at least 88% identical to, such at least 89% identical to, such at least 90% identical to, such at least 91% identical to, such at least 92% identical to, such at least 93% identical to, such at least 94% identical to, such at least 95% identical to, such at least 96% identical to, such at least 97% identical to, such at least 98% identical to, such at least 99% identical to, such at least 99.5% identical to, such at least 99.6% identical to, such at least 99.7% identical to, such at least 99.8% identical to, such at least 99.9% identical to said SEQ ID NO: 13, SEQ ID NO: 33, SEQ ID NO: 26, SEQ ID NO: 34, SEQ ID NO: 4, SEQ ID NO: 5, SEQ ID NO: 6, SEQ ID NO: 7, SEQ ID NO: 8, SEQ ID NO: 8, SEQ ID NO: 9, SEQ ID NO: 10, SEQ ID NO: 11, SEQ ID NO: 12, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 27, SEQ ID NO: 28, SEQ ID NO: 29 or SEQ ID NO: 30, and wherein the biological activity is capability to inhibit the prolactin receptor signalling; or
iii) a biologically active fragment of i) or ii) wherein said fragment comprises at least 50 contiguous amino acids, such as at least 60 contiguous amino acids, such as at least 70 contiguous amino acids, such as at least 80 contiguous amino acids, such as at least 90 contiguous amino acids, such as at least 100 contiguous amino acids, such as at least 110 contiguous amino acids, such as at least 120 contiguous amino acids, such as at least 130 contiguous amino acids, such as at least 140 contiguous amino acids, such as at least 150 contiguous amino acids, such as at least 160 contiguous amino acids of any one of said SEQ ID NO: 13, SEQ ID NO: 33, SEQ ID NO: 26, SEQ ID NO: 34, SEQ ID NO: 4, SEQ ID NO: 5, SEQ ID NO: 6, SEQ ID NO: 7, SEQ ID NO: 8, SEQ ID NO: 8, SEQ ID NO: 9, SEQ ID NO: 10, SEQ ID NO: 11, SEQ ID NO: 12, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 27, SEQ ID NO: 28, SEQ ID NO: 29 and SEQ ID NO: 30, wherein the biological activity is capability to inhibit the prolactin receptor; or
b) a polynucleotide encoding the polypeptide of a), or
c) a vector comprising the polynucleotide of b), or
d) a host cell comprising the polynucleotide of b) and/or the vector of c).

6. The prolactin receptor antagonist for use according to any one of the preceding claims, wherein the prolactin receptor antagonist is a polypeptide, wherein said polypeptide further comprises a chemically conjugated entity capable of increasing the half-life of the prolactin receptor antagonist when administered to a patient, in particular its plasma and/or serum half-life.

7. The prolactin receptor antagonist for use according to any one of the preceding claims, wherein the prolactin receptor antagonist is a polypeptide, wherein said polypeptide further comprises a moiety conjugated to said antagonist, thus generating a moiety-conjugated antagonist.

8. The prolactin receptor antagonist for use according to any one of the preceding claims, wherein the prolactin receptor antagonist is a polypeptide, wherein said polypeptide further comprises, wherein the moiety-conjugated antagonist has a plasma and/or serum half-life being longer than the plasma and/or serum half-life of the non-moiety conjugated agent.

9. The prolactin receptor antagonist for use according to any one of the preceding claims, wherein the prolactin receptor antagonist is a polypeptide, wherein said polypeptide has been conjugated with a moiety facilitating crossing of the blood-brain-barrier, such as wherein the moiety is an antibody from a camelid species such as a recombinant or native single-chain antibody from dromedaries, camels, llamas, alpacas, vicuñas, or guanacos.

10. The prolactin receptor antagonist for use according to any one of the preceding claims, wherein the prolactin receptor antagonist is a polypeptide, wherein said polypeptide further comprises a moiety conjugated to the antagonist wherein the moiety is one or more type of moieties selected from the group consisting of albumin, fatty acids, polyethylene glycol (PEG), acylation groups, antibodies and antibody fragments.

11. The prolactin receptor antagonist for use according to any one of the preceding claims, wherein the prolactin receptor antagonist is a polypeptide, wherein said polypeptide further comprises an N-terminal-albumin binding peptide, wherein said N-terminal-albumin binding peptide is CPGPPGS (SEQ ID NO 31).

12. The prolactin receptor antagonist for use according to any one of the preceding claims, wherein the prolactin receptor antagonist is a polypeptide, wherein said polypeptide further comprises an N-terminal-albumin binding peptide, wherein said N-terminal-albumin binding peptide is DDEWLCGWRPLCIDEILRPGPPGS (SEQ ID NO 32).

13. The prolactin receptor antagonist for use according to any one of the preceding claims, wherein the prolactin receptor antagonist is a polypeptide, wherein said polypeptide further comprises an at least one bis-maleimide containing linker.

14. The prolactin receptor antagonist for use according to any one of the preceding claims, wherein the prolactin receptor antagonist is a polypeptide, wherein said polypeptide further comprises at least two bis-maleimide containing linkers, such as wherein the linker is (BML)(BML)-(CPGPPGS), e.g. an N-terminally conjugated linked (BML)(BML)-(CPGPPGS).

15. The prolactin receptor antagonist according to any one of the preceding claims, wherein the prolactin receptor antagonist is a polypeptide, wherein said polypeptide further comprises at least two bis-maleimide containing linkers, wherein said linker is N-terminally linked to said SEQ ID NO: 13, SEQ ID NO: 33, SEQ ID NO: 26, SEQ ID NO: 34, SEQ ID NO: 4, SEQ ID NO: 5, SEQ ID NO: 6, SEQ ID NO: 7, SEQ ID NO: 8, SEQ ID NO: 8, SEQ ID NO: 9, SEQ ID NO: 10, SEQ ID NO: 11, SEQ ID NO: 12, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 27, SEQ ID NO: 28, SEQ ID NO: 29 or SEQ ID NO: 30.

16. The prolactin receptor antagonist for use according to any one of the preceding claims, wherein the prolactin receptor antagonist is a polypeptide, wherein said polypeptide further comprises a Bis-maleimid PEG linker, such as a sequence selected from the group consisting of SEQ ID NO: 13, SEQ ID NO: 33, SEQ ID NO: 26, SEQ ID NO: 34, SEQ ID NO: 4, SEQ ID NO: 5, SEQ ID NO: 6, SEQ ID NO: 7, SEQ ID NO: 8, SEQ ID NO: 8, SEQ ID NO: 9, SEQ ID NO: 10, SEQ ID NO: 11, SEQ ID NO: 12, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 27, SEQ ID NO: 28, SEQ ID NO: 29 and SEQ ID NO: 30, wherein said linker is N-terminally linked to said SEQ ID NO: 13, SEQ ID NO: 33, SEQ ID NO: 26, SEQ ID NO: 34, SEQ ID NO: 4, SEQ ID NO: 5, SEQ ID NO: 6, SEQ ID NO: 7, SEQ ID NO: 8, SEQ ID NO: 8, SEQ ID NO: 9, SEQ ID NO: 10, SEQ ID NO: 11, SEQ ID NO: 12, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 27, SEQ ID NO: 28, SEQ ID NO: 29 and SEQ ID NO: 30.

17. The prolactin receptor antagonist for use according to any one of the preceding claims, wherein the prolactin receptor antagonist is a polypeptide, wherein said polypeptide further comprises a tag, such as a polyhis tag, a GST tag, a HA tag, a Flag tag, a C-myc tag, a HSV tag, a V5 tag, a maltose binding protein tag, a cellulose binding domain tag.

18. The prolactin receptor antagonist for use according to any one of the preceding claims, wherein the polypeptide is glycosylated.

19. The prolactin receptor antagonist for use according to any one of the preceding claims, wherein the prolactin receptor antagonist is a polypeptide capable of forming at least one intramolecular cystine bridge.

20. The prolactin receptor antagonist according for use to any one of the preceding claims, wherein the prolactin receptor antagonist is a polypeptide comprising a dimer of said polypeptide, linked through at least one intermolecular cystine bridge.

21. The prolactin receptor antagonist for use according to any one of the preceding claims wherein said antagonist is administered simultaneously with, immediately subsequent to, or immediately prior to, a further active ingredient.

22. The prolactin receptor antagonist for use according to any one of the preceding claims wherein said second or further active ingredient is capable of inhibiting growth of glioblastomas.

23. The prolactin receptor antagonist for use according to any one of the preceding claims wherein said second or further active ingredient is selected from the group consisting of growth factor antagonists, kinase inhibitors and anti-mitotic chemotherapeutics.

24. The prolactin receptor antagonist for use according to any one of the preceding claims wherein said second or further active ingredient is selected from the group consisting of Temezolomide, Bevacizumab and compounds targeting the EGF receptor or its signal transduction, compounds targeting PDGF or its signal transduction, compounds targeting HDAC, compounds targeting mTOR such as. Sirolimus, compounds for treatments based on cell therapy such as dendritic cell vaccination or wherein the second or further compound is antiviral compounds such as Ganciclorvir.

25. The prolactin receptor antagonist for use according to any one of the preceding claims wherein said compound is combined with radiation therapy or agents facilitating effects of radiation therapy such as Docitaxel or vitamin D.

26. The prolactin receptor antagonist for use according to any one of the preceding claims, wherein said antagonist is formulated as a pharmaceutical composition suitable for enteral or parenteral administration.

27. The prolactin receptor antagonist for use according to any one of the preceding claims, wherein said antagonist is formulated as a pharmaceutical composition suitable for parenteral administration, such as subcutaneous, intrathecal, intraspinal, intraperitoneal, intravenous, intramuscular, a bolus or for continuous administration.

28. The prolactin receptor antagonist for use according to any one of the preceding claims, wherein said antagonist is administered locally at the site of a tumor.

29. The prolactin receptor antagonist for use according to any one of the preceding claims, wherein the prolactin receptor antagonist is an anti-prolactin receptor antibody.

30. The prolactin receptor antagonist for use according to any one of the preceding claims, wherein the prolactin receptor antagonist is an antibody selected from the group consisting of: polyclonal antibodies, monoclonal antibodies, humanised antibodies, single chain antibodies, and recombinant antibodies.

31. The prolactin receptor antagonist for use according to any one of the preceding claims, wherein said antagonist comprises or consists of an antibody or an antigen-binding fragment thereof with binding specificity for the prolactin receptor, or a variant, fusion or derivative of said antibody or antigen-binding fragment, or a fusion of a said variant or derivative thereof, which retains the binding specificity for a prolactin receptor.

32. The prolactin receptor antagonist according to any one of the preceding claims, further comprising a cytotoxic moiety.

33. The prolactin receptor antagonist according to any one of the preceding claims, further comprising a cytotoxic moiety, wherein the cytotoxic moiety comprises or consists of a radioisotope.

34. The prolactin receptor antagonist according to any one of the preceding claims, further comprising a cytotoxic moiety, wherein the cytotoxic moiety comprises or consists of a radioisotope, wherein the radioisotope is selected from the group consisting of astatine-211, bismuth-212, bismuth-213, iodine-131, yttrium-90, lutetium-177, samarium-153 and palladium-109.

35. The prolactin receptor antagonist according to any one of the preceding claims, further comprising a cytotoxic moiety, wherein the cytotoxic moiety comprises or consists of a toxin (such as saporin or calicheamicin).

36. The prolactin receptor antagonist according to any one of the preceding claims, further comprising a cytotoxic moiety, wherein the cytotoxic moiety comprises or consists of a chemotherapeutic agent.

37. The prolactin receptor antagonist according to any one of the preceding claims, further comprising a detectable moiety.

38. The prolactin receptor antagonist according to any one of the preceding claims, further comprising a detectable moiety, wherein the detectable moiety comprises or consists of a radioisotope.

39. The prolactin receptor antagonist according to any one of the preceding claims, further comprising a detectable moiety, wherein the detectable moiety comprises or consists of a radioisotope selected from the group consisting of technitium-99m, indium-111, gallium-67, gallium-68, arsenic-72, zirconium-89, iodine-12, thallium-201.

40. The prolactin receptor antagonist according to any one of the preceding claims, further comprising a detectable moiety, wherein the detectable moiety comprises or consists of a paramagnetic isotope.

41. The prolactin receptor antagonist according to any one of the preceding claims, further comprising a detectable moiety, wherein the detectable moiety comprises or consists of a paramagnetic isotope wherein the paramagnetic isotope is selected from the group consisting of gadolinium-157, manganese-55, dysprosium-162, chromium-52, and iron-56.

42. The prolactin receptor antagonist for use according to any one of the preceding claims wherein said antagonist is administered simultaneously with, immediately subsequent to, or immediately prior to, a second or further active ingredient, wherein said second or further active ingredient is capable of inhibiting growth of glioblastomas.

43. A method of treatment of glioblastomas of a mammal in need thereof, the method comprising the steps of:
a) obtaining tissue samples of a glioblastoma, and
b) analyzing said sample for presence of Prl receptors,
c) comparing said sample to a control sample from healthy tissue,
d) determining sensitivity of the mammal to treatment with a prolactin receptor antagonist according to any one of the preceding claims,
e) administering a therapeutically effective amount of said prolactin receptor antagonist defined in any one of the preceding claims.

44. A method of inducing cell death in a tumor cell expressing a prolactin receptor, said method comprising administering a prolactin receptor antagonist to a patient diagnosed with a neoplasm of the brain or spinal cord.

45. A method of inhibiting growth and/or invasion and/or proliferation of tumor cells, the method comprising administering a prolactin receptor antagonist to a patient in need thereof.


SEQUENCE LISTING (34 sequences)

SEQ ID NO: 1 - protein, 622 aa, Homo Sapiens
MKENVASATVFTLLLFLNTCLLNGQLPPGKPEIFKCRSPNKETFTCWWRPGTDGGLPTNYSLTYHREGETLMHECPDYITGGPNSCHFGKQYTSMWRTYIMMVNATNQMGSSFSDELYVDVTYIVQPDPPLELAVEVKQPEDRKPYLWIKWSPPTLIDLKTGWFTLLYEIRLKPEKAAEWEIHFAGQQTEFKILSLHPGQKYLVQVRCKPDHGYWSAWSPATFIQIPSDFTMNDTTVWISVAVLSAVICLIIVWAVALKGYSMVTCIFPPVPGPKIKGFDAHLLEKGKSEELLSALGCQDFPPTSDYEDLLVEYLEVDDSEDQHLMSVHSKEHPSQGMKPTYLDPDTDSGRGSCDSPSLLSEKCEEPQANPSTFYDPEVIEKPENPETTHTWDPQCISMEGKIPYFHAGGSKCSTWPLPQPSQHNPRSSYHNITDVCELAVGPAGAPATLLNEAGKDALKSSQTIKSREEGKATQQREVESFHSETDQDTPWLLPQEKTPFGSAKPLDYVEIHKVNKDGALSLLPKQRENSGKPKKPGTPENNKEYAKVSGVMDNNILVLVPDPHAKNVACFEESAKEAPPSLEQNQAEKALANFTATSSKCRLQLGGLDYLDPACFTHSFH

SEQ ID NO: 2 - protein, 227 aa, Homo Sapiens
MNIKGSPWKGSLLLLLVSNLLLCQSVAPLPICPGGAARCQVTLRDLFDRAVVLSHYIHNLSSEMFSEFDKRYTHGRGFITKAINSCHTSSLATPEDKEQAQQMNQKDFLSLIVSILRSWNEPLYHLVTEVRGMQEAPEAILSKAVEIEEQTKRLLEGMELIVSQVHPETKENEIYPVWSGLPSLQMADEESRLSAYYNLLHCLRRDSHKIDNYLKLLKCRIIHNNNC

SEQ ID NO: 3 - protein, 199 aa, Homo Sapiens
LPICPGGAARCQVTLRDLFDRAVVLSHYIHNLSSEMFSEFDKRYTHGRGFITKAINSCHTSSLATPEDKEQAQQMNQKDFLSLIVSILRSWNEPLYHLVTEVRGMQEAPEAILSKAVEIEEQTKRLLEGMELIVSQVHPETKENEIYPVWSGLPSLQMADEESRLSAYYNLLHCLRRDSHKIDNYLKLLKCRIIHNNNC

SEQ ID NO: 4 - protein, 199 aa, Homo Sapiens
LPICPGGAARCQVTLRDLFDRAVVLSHYIHNLASEMFSEFDKRYTHGRGFITKAINSCHTSSLATPEDKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 5 - protein, 198 aa, Homo Sapiens
PICPGGAARCQVTLRDLFDRAVVLSHYIHNLASEMFSEFDKRYTHGRGFITKAINSCHTSSLATPEDKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 6 - protein, 197 aa, Homo Sapiens
ICPGGAARCQVTLRDLFDRAVVLSHYIHNLASEMFSEFDKRYTHGRGFITKAINSCHTSSLATPEDKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 7 - protein, 196 aa, Homo Sapiens
CPGGAARCQVTLRDLFDRAVVLSHYIHNLASEMFSEFDKRYTHGRGFITKAINSCHTSSLATPEDKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 8 - protein, 195 aa, Homo Sapiens
PGGAARCQVTLRDLFDRAVVLSHYIHNLASEMFSEFDKRYTHGRGFITKAINSCHTSSLATPEDKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 9 - protein, 194 aa, Homo Sapiens
GGAARCQVTLRDLFDRAVVLSHYIHNLASEMFSEFDKRYTHGRGFITKAINSCHTSSLATPEDKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 10 - protein, 193 aa, Homo Sapiens
GAARCQVTLRDLFDRAVVLSHYIHNLASEMFSEFDKRYTHGRGFITKAINSCHTSSLATPEDKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 11 - protein, 192 aa, Homo Sapiens
AARCQVTLRDLFDRAVVLSHYIHNLASEMFSEFDKRYTHGRGFITKAINSCHTSSLATPEDKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 12 - protein, 191 aa, Homo Sapiens
ARCQVTLRDLFDRAVVLSHYIHNLASEMFSEFDKRYTHGRGFITKAINSCHTSSLATPEDKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 13 - protein, 190 aa, Homo Sapiens
RCQVTLRDLFDRAVVLSHYIHNLASEMFSEFDKRYTHGRGFITKAINSCHTSSLATPEDKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 14 - protein, 189 aa, Homo Sapiens
CQVTLRDLFDRAVVLSHYIHNLASEMFSEFDKRYTHGRGFITKAINSCHTSSLATPEDKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 15 - protein, 188 aa, Homo Sapiens
QVTLRDLFDRAVVLSHYIHNLASEMFSEFDKRYTHGRGFITKAINSCHTSSLATPEDKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 16 - protein, 187 aa, Homo Sapiens
VTLRDLFDRAVVLSHYIHNLASEMFSEFDKRYTHGRGFITKAINSCHTSSLATPEDKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 17 - protein, 199 aa, Homo Sapiens
LPICPGGAARCQVTLRDLFDRAVVLSHYIHNLSSEMFSEFDKRYTHGRGFITKAINSCHTASLATPENKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 18 - protein, 198 aa, Homo Sapiens
PICPGGAARCQVTLRDLFDRAVVLSHYIHNLSSEMFSEFDKRYTHGRGFITKAINSCHTASLATPENKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 19 - protein, 197 aa, Homo Sapiens
ICPGGAARCQVTLRDLFDRAVVLSHYIHNLSSEMFSEFDKRYTHGRGFITKAINSCHTASLATPENKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 20 - protein, 196 aa, Homo Sapiens
CPGGAARCQVTLRDLFDRAVVLSHYIHNLSSEMFSEFDKRYTHGRGFITKAINSCHTASLATPENKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 21 - protein, 195 aa, Homo Sapiens
PGGAARCQVTLRDLFDRAVVLSHYIHNLSSEMFSEFDKRYTHGRGFITKAINSCHTASLATPENKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 22 - protein, 194 aa, Homo Sapiens
GGAARCQVTLRDLFDRAVVLSHYIHNLSSEMFSEFDKRYTHGRGFITKAINSCHTASLATPENKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 23 - protein, 193 aa, Homo Sapiens
GAARCQVTLRDLFDRAVVLSHYIHNLSSEMFSEFDKRYTHGRGFITKAINSCHTASLATPENKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 24 - protein, 192 aa, Homo Sapiens
AARCQVTLRDLFDRAVVLSHYIHNLSSEMFSEFDKRYTHGRGFITKAINSCHTASLATPENKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 25 - protein, 191 aa, Homo Sapiens
ARCQVTLRDLFDRAVVLSHYIHNLSSEMFSEFDKRYTHGRGFITKAINSCHTASLATPENKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 26 - protein, 190 aa, Homo Sapiens
RCQVTLRDLFDRAVVLSHYIHNLSSEMFSEFDKRYTHGRGFITKAINSCHTASLATPENKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 27 - protein, 189 aa, Homo Sapiens
CQVTLRDLFDRAVVLSHYIHNLSSEMFSEFDKRYTHGRGFITKAINSCHTASLATPENKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 28 - protein, 188 aa, Homo Sapiens
QVTLRDLFDRAVVLSHYIHNLSSEMFSEFDKRYTHGRGFITKAINSCHTASLATPENKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 29 - protein, 187 aa, Homo Sapiens
VTLRDLFDRAVVLSHYIHNLSSEMFSEFDKRYTHGRGFITKAINSCHTASLATPENKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 30 - protein, 188 aa, Homo Sapiens
SVTLRDLFDRAVVLSHYIHNLSSEMFSEFDKRYTHGRGFITKAINSCHTASLATPENKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 31 - protein, 7 aa, Artificial Sequence
  N-terminal tag
CPGPPGS

SEQ ID NO: 32 - protein, 24 aa, Artificial Sequence
  N-terminal tag
DDEWLCGWRPLCIDEILRPGPPGS

SEQ ID NO: 33 - protein, 191 aa, Homo sapiens
SRCQVTLRDLFDRAVVLSHYIHNLASEMFSEFDKRYTHGRGFITKAINSCHTSSLATPEDKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC

SEQ ID NO: 34 - protein, 191 aa, Homo sapiens
SRCQVTLRDLFDRAVVLSHYIHNLSSEMFSEFDKRYTHGRGFITKAINSCHTASLATPENKEQALQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLRCRIIHNNNC